(19) United States
(12) Patent Application Publication
(10) Pub. No.: US 2021/0361611 A1
(43) Pub. Date: Nov. 25, 2021

(54) TREATMENT OF DISORDERS WITH TASIMELTEON

(71) Applicant: Vanda Pharmaceuticals Inc., Washington, DC (US)

(72) Inventors: Vasilios POLYMEROPOULOS, Potomac, MD (US); Christos POLYMEROPOULOS, Potomac, MD (US); Changfu XIAO, Vienna, VA (US); Mihael H. POLYMEROPOULOS, Potomac, MD (US)

(21) Appl. No.: 16/977,801

(22) PCT Filed: Mar. 4, 2019
(86) PCT No.: PCT/US2019/020491

§ 371 Date: Sep. 2, 2020

Related U.S. Application Data
(60) Provisional application No. 62/675,687, filed on May 23, 2018.
(60) Provisional application No. 62/638,212, filed on Mar. 4, 2018.

(51) Int. Cl. A61K 31/343; A61P 25/20; A61J 1/03

(52) U.S. Cl. CPC A61K 31/343; A61J 1/035; A61P 25/20

(57) ABSTRACT

Tasimelteon improves sleep in individuals experiencing an advance in established bedtime.

### CROSS-REFERENCE TO RELATED APPLICATIONS

This application claims the benefit of co-pending U.S. Provisional Patent Application Ser. No. 62/638,212, filed 4 Mar. 2018 and 62/675,687, filed 23 May 2018, each of which is incorporated herein as though fully set forth.

### BACKGROUND OF THE INVENTION

The present invention relates to the clinical use of tasimelteon to treat disorders, specifically disorders arising from an abruptly advanced circadian rhythm as a result of a change in an individual's normal bedtime. Such changes in normal bedtime can result from the need to adapt to a new sunrise-sunset cycle, such as in the case of travel across multiple time zones that creates a need to rapidly adapt to a new local time at the destination of the travel, or to an imposed bedtime advance, as in the case of a “shift change” in which, for example, a day-shift worker begins a night-shift job (e.g., resulting in the worker's established 8:00 a.m. shift start time changing to a midnight shift start time and producing a bedtime advance from 11:00 p.m. to 3:00 p.m. in the worker's time zone).

The disorder resulting from an abruptly advanced circadian rhythm arising from travel across multiple time zones is commonly referred to as “jet lag disorder” or “JLD” and is commonly characterized by nighttime sleep disruption and daytime decrease in alertness. JLD is associated with disruption in social and occupational functioning and affects millions of individuals annually, with symptoms that are often more severe during eastward travel. It is estimated that globally, over 100 million people travel across five or more time zones annually.

Tasimelteon, also referred to as MA-1, HETLIOZ®, and pharmaceutical compositions and uses thereof have been described in the art. Tasimelteon is approved for use as a human medicine for the treatment of Non-24-Hour Sleep-Wake Disorder (Non-24) and is available in a 20 mg unit pharmaceutical dosage form (capsules), indicated for use prior to bedtime at the same time every night. Pharmacologically, tasimelteon is an agonist of the MT1R and MT2R melatonin receptors in the Suprachiasmatic nucleus (SCN), the region of the brain associated with the biological clock. Engagement of these receptors by melatonin is believed to regulate circadian rhythms, including the sleep/wake cycle. Consistent with its receptor binding profile, tasimelteon demonstrates potent chronobiotic activity in preclinical models of acute phase-shifting and chronic re-entrainment.

Tasimelteon per se is claimed in U.S. Pat. No. 5,856,529 in claim 7 thereof. The '529 patent contains further claims, including claims to a genus of compounds of which tasimelteon is a member, as well as claims to the use of this genus in treating sleep disorders, as well as circadian rhythm disorders, in patients by administering an effective amount of tasimelteon. The patent describes tasimelteon as a melatonin agonist and further speculates that melatonin agonists would be useful for the further study of melatonin receptor interactions as well as in the treatment of conditions affected by melatonin activity. The patent lists depression, jet lag, work-shift syndrome, and sleep disorders, among other possible therapeutic uses. Elsewhere the patent discloses that compounds within genus of compounds of which tasimelteon is a member are useful as melatonergic agents in the treatment of sleep disorders, seasonal depression, shifts in circadian cycles, melancholia, stress, appetite regulation, benign prostatic hyperplasia and related conditions.

In addition to tasimelteon's approved dosing of 20 mg per day prior to bedtime at the same time every day, WO2007/137244 reports the discovery that effective human doses for tasimelteon can range from 10 to 100 mg/day for contemplated uses in sleep disorders and circadian rhythm disorders, with a further description that the exact dosing may be dependent upon particle size of the tasimelteon and the body size of the patient being treated. The patent describes also describes a 20 mg oral unit dosage form for tasimelteon and a clinical trial using tasimelteon in 10 mg, 20 mg, 50 mg, and 100 mg daily doses.

In U.S. Published Application Publication 20090105333A1 (WO2007/137244), results are reported from the aforementioned clinical trial in which tasimelteon was studied in subjects with a 5-hour advance in their sleep-wake cycle, i.e., the type of sleep-wake cycle advance that might be experienced by a subject traveling by jet aircraft across the Atlantic Ocean from New York to London, including that treatment relative to placebo produced positive outcomes for shifting dim light melatonin onset and sleep efficacy. The description of this trial and its reported outcomes is incorporated herein by reference as though fully set forth.

### SUMMARY OF THE INVENTION

The present invention particularly includes a method of treating an individual experiencing sleep-wake-cycle-disrupting advance in the individual's established or normal bedtime comprising administering to the individual an amount of tasimelteon effective to reduce one or more untoward consequences of the resulting disruption of said individual's established sleep-wake cycle. The invention includes, therefore, an advance of up to nine hours in the individual's established bedtime, although a similar disruption can take place with an advance of up to eight hours, e.g., a six- to eight-hour advance.

The amount of tasimelteon administered is typically 20 mg per day. Ideally, the tasimelteon is administered in an immediate release form for at least three consecutive days in treating the advance. For example, a disruption producing an advance of eight hours in an individual's established bedtime can be treated with tasimelteon administration for five consecutive days.

Optimally, the tasimelteon is administered prior to bedtime, i.e., at a time proximate to the individual's bedtime. Typically, tasimelteon is administered 30 minutes to one hour prior to bedtime, but alternatively may be administered up to two hours prior to bedtime. In treating the advance, the first dose of the at least three doses is administered prior to the first bedtime following the sleep-wake-cycle-disrupting advance.

When tasimelteon is administered according to the regimen above, the reduction of untoward consequences includes an improvement in at least one sleep parameter selected from a group consisting of total sleep time, latency to persistent sleep, and wake after sleep onset, e.g., improvement in total sleep time, specifically an improvement in the first two-thirds of the night on the third night following tasimelteon administration, or an improvement in next day alertness, which may be measured as an improvement on the Karolinska Sleepiness Scale, an improvement on the Visual Analog Scale, or both.

Thus, the present invention includes treatment of an individual where the advance in the individual's established bedtime is up to nine hours (e.g., six to eight hours) and the amount of tasimelteon administered is 20 mg per day, administered for at least three consecutive days prior to bedtime, with the first dose administered in an immediate release form prior to the first bedtime following the sleep-wake-cycle-disrupting advance. In this regard, one aspect of the method treats an individual who experiences a sleep-wake-cycle-disrupting advance as a result of eastbound jet aircraft travel. Such individual, for example, may experience a significantly improved total sleep time during the first two-thirds of the night, during the night following the administration of the third dose of tasimelteon, in both subjective and objective measures of sleep.

Another aspect of the present invention is the treatment of an individual who is known, from the individual's medical history, to have experienced a prior sleep-wake-cycle-disrupting advance and, therefore, is known to be in need of therapy to reduce one or more untoward consequences of a subsequent disruption of said individual's established sleep-wake cycle. For example, the individual may be known to experience untoward consequences of a disruption of said individual's established sleep-wake cycle from eastbound jet aircraft travel.

One aspect of the above method arises when the sleep-wake-cycle-disrupting advance occurs upon arrival, following an eastbound jet aircraft flight, in which clock time is advanced at the destination of up to eight hours (e.g., six to eight hours) relative to the clock time at the origin of the flight. Specifically, the present invention includes the treatment of an individual who is known, from the individual's medical history of a prior sleep-wake-cycle-disrupting advance, to be in need of therapy to reduce one or more untoward consequences of a subsequent disruption of said individual's established sleep-wake cycle. Thus, according to the method described above, such an individual may be administered 20 mg of tasimelteon per day, administered in an immediate release form for at least three consecutive days up to one hour prior to bedtime, with the first dose administered prior to the first bedtime following the sleep-wake-cycle-disrupting advance.

An aspect of the invention is the treatment of individuals who experience at least one night of sleep disruption prior to experiencing the six- to eight-hour bedtime advance. An example of sleep disruption would be at least one waking during the normal non-advanced individual's night, such as when landing in London after an overnight flight from the East or West coasts of the United States.

One specific aspect of the present invention involves a method of treating jet lag in an individual subjected to eastward travel from a place of origin through six to eight time zones (e.g., through eight time zones, such as would be experienced during an eastward flight from San Francisco to London) to a place of destination during overnight travel by air, said method comprising orally administering to the individual 20 mg tasimelteon in immediate release form following arrival at the place of destination at or prior to the individual's bedtime in the place of destination. Such method includes administration up to 30 minutes to up to two hours prior to said individual's bedtime corresponding to the clock time (i.e., local time) of the individual's regular bedtime at his or her place of origin (e.g., at, or within 30, 60, 90, or 120 minutes of, such bedtime), and includes administration to individuals who are pre-selected for tasimelteon administration based on having suffered jet lag disorder as a result of previous similar travel experience and who are treated by administration of tasimelteon once daily for at least three consecutive days (e.g., for up to five consecutive days).

Another aspect of the invention provides a method of treating an individual experiencing a sleep-wake-cycle-disrupting advance in the individual's established bedtime of up to eight hours comprising administering to the individual 20 mg of tasimelteon per day, administered for at least three consecutive days, up to one hour prior to the individual's advanced (i.e., local) bedtime, with the first dose administered prior to the first bedtime following the sleep-wake-cycle-disrupting advance.

The present invention may, therefore, involve either reducing an untoward consequence of an advance of up to nine hours (e.g., 6 to 8 hours) in an individual's established bedtime, or otherwise treating an individual experiencing a sleep-wake-cycle-disrupting advance in the individual's established bedtime of up to eight hours, by administering to the individual 20 mg of tasimelteon, in an immediate release form, prior to the individual's advanced bedtime, for at least three consecutive days, with the first dose administered prior to the first advanced bedtime.

As used herein, in the context of a sleep-wake-cycle-disrupting advance caused by eastward jet travel, an individual's “advanced bedtime” is the individual's regular or established bedtime in the new, eastern location. For example, an individual having a regular bedtime of 10:00 PM in Los Angeles would have an “advanced bedtime” of 10:00 PM in London.

In general, the present invention relates to a sleep-wake-cycle-disrupting advance in an individual's established sleep-wake cycle that arises when an individual's normal or established bedtime is abruptly advanced to a sufficiently earlier time (relative to the individual's established 24-hour clock) that the individual experiences one or more of the known consequences of this type of disruption of the individual's sleep wake cycle.

For example, on a jet aircraft flight from San Francisco to London, an individual arriving in London would experience an approximately 8-hour advance in the individual's established sleep-wake cycle given the individual's transit across eight time zones. Lesser or greater advances in an individual's established bedtime are likewise known to produce untoward consequences from such a sleep-wake cycle disturbance. Such an advance would occur over the duration of the travel, e.g., 10 to 13 hours.

Amounts of tasimelteon effective in the above method are known in the art from the clinical studies of tasimelteon previously reported. For example, a 20 mg capsule of tasimelteon is useful in the above method and can be administered at or preferable before the advanced bedtime of the individual, e.g., 30 minutes to two hours prior to the advanced bedtime.

The reduction in untoward consequences from a sleep-wake-cycle-disrupting advance may be determined by the individual's response to treatment, such as through improved sleep parameters, such as improved total sleep time, improved sleep time during the first two-thirds of the night, increased rapid eye movement (REM) sleep, and/or a reduction in the time to accumulate 30 minutes of REM sleep, as compared to what the individual would have experienced in the absence of tasimelteon treatment.

Measures of direct improvement include higher next-day alertness, which can be measured using established tools, such as improvement on the Karolinska Sleepiness Scale or the Visual Analog Scale, or both.

Finally, the invention herein includes a pre-packaged dispensing unit containing a number of individual tasimelteon unit doses (e.g., tablets), each containing 20 mg of tasimelteon, sufficient to provide a course of treatment for an individual to be treated for a jet lag disorder, e.g., three to five such immediate release 20 mg tasimelteon tablets per individual dispensing unit. Such dispensing unit can comprise any of the conventional pharmaceutical containers for medicines being dispensed. Particular preferred dispensing units are 3-unit dose or 5-unit dose blister packs.

### EXAMPLES

Aspects of the invention are more fully understood based from the clinical trial results described in Examples 1 and 2, as set out below.

### Example 1

A Phase III clinical study of 318 healthy volunteers demonstrates the efficacy of tasimelteon in treating jet lag disorder. In the course of the trial, volunteers are subjected to a circadian challenge of an 8-hour advance in their usual bedtime, consistent with the circadian challenge induced in travelers crossing eight time zones (e.g., Los Angeles to London or Washington, D.C. to Moscow) and typically causing jet lag disorder.

The results of this study demonstrate highly significant and clinically meaningful effects of a 20 mg dose of tasimelteon on the primary endpoint of the study, as well as multiple secondary endpoints. The pre-specified primary endpoint used for this purpose is the amount of sleep time in the first two thirds of the night. Secondary endpoints include measures of various sleep parameters—total sleep time (TST), latency to persistent sleep (LPS), and wake after sleep onset (WASO)—as well as next day alertness, measured using the Karolinska Sleepiness Scale (KSS) and Visual Analog Scale (VAS). Table 1 below provides a summary of the primary and secondary endpoint results.

TABLE 1

| | | | | | p-value | p-value |
|---|---|---|---|---|---|---|
| Assessment | Endpoint | Hetlioz ® | Placebo | Diff | Summary | Detail |
| PSG | TST_{2/3}* | 216.4 | 156.1 | 60.31 | P < 0.001 | 3.29E−12 |
| (minutes) | TST_{full} | 315.8 | 230.3 | 85.46 | P < 0.001 | 3.74E−14 |
| | LPS | 21.8 | 36.8 | −15.08 | P < 0.01 | 8.08E−03 |
| | WASO | 144.6 | 219.1 | −74.58 | P < 0.001 | 3.41E−12 |
| KSS (1-9) | average | 4.0 | 4.5 | −0.53 | P < 0.01 | 8.28E−03 |
| VAS (0-100) | average | 60.8 | 54.2 | 6.59 | P < 0.01 | 9.89E−03 |

*primary endpoint

These results demonstrate the effectiveness of tasimelteon in treating jet lag disorder. The magnitude of the total sleep time benefit of 85 minutes' improvement over placebo is highly clinically meaningful. The measurements of next day alertness on both the KSS and VAS scales are also meaningful and underscore the ability of tasimelteon to address both nighttime and daytime symptoms of jet lag disorder.

Tasimelteon was also shown to significantly increase the total rapid eye movement (REM) sleep period while also significantly decreasing the time required to accumulate 30 minutes of REM sleep. These results are shown in Table 2 below.

TABLE 2

| Assess- | | | | | p-value |
|---|---|---|---|---|---|
| ment | Endpoint | Hetlioz ® | Placebo | Difference | Summary |
| PSG | REM_{Total} | 48.9 | 35.2 | 13.7 | <0.0001 |
| (min.) | | | | | |
| (min.) | REM_{Accumulate} | 318.7 | 372 | −53.3 | <0.0001 |
| (%) | REM_{Achieved} | 76.1 | 51.6 | 24.5 | <0.001 |

Thus, tasimelteon demonstrated significant improvement in the accumulation of REM sleep, which is strongly regulated by the circadian pacemaker, during an 8-hour phase advance in sleep timing. These results suggest that tasimelteon increases sleep during circadian adverse timing at least in party by affecting the circadian pacemaker and further support tasimelteon as a novel circadian regulator for the treatment of JLD.

### Example 2

A two-phase transatlantic travel study of 25 subjects (tasimelteon n=13, placebo n=12) comprises a first phase that is an observational travel study to collect baseline data, and a second phase that is a treatment phase. Participants in the study travel either five or eight time zones from Washington, D.C. to London (five time zones) or San Francisco or Los Angeles to London (eight time zones). Participants stay at their destination for three nights and four days and during randomization receive 20 mg of tasimelteon for three consecutive nights prior to their bedtime. Efficacy is monitored by PSG as well as sleep and wake questionnaire scales.

During the baseline phase, sleep is most disturbed in the third night, as shown in Table 3 below.

TABLE 3
Baseline TST_{2/3} (minutes)

| Night 1 | Night 2 | Night 3 |
|---|---|---|
| 217.8 (59.78) | 249.8 (51.62) | 197.5 (88.76) |

The primary endpoint of the study is TST for the first ⅔ of the night(s) most likely to be disrupted. These are, from Table 2, Night 3, followed by Night 1 and Night 2.

Table 4 below reports the effect of tasimelteon versus placebo on TST_{2/3} and TST_{full} on Night 3, as well as measures of sleep quality, sleep latency, and WASO.

TABLE 4

| | | Change from baseline | | | |
|---|---|---|---|---|---|
| | Endpoint | HETLIOZ ® | Placebo | Difference | p-value |
| Objective | TST_{2/3} Night 3 | 76.2 | 41.4 | 34.8 | p = 0.0354 |
| | TST_{2/3} All 3 nights | 131.4 | 40.9 | 90.6 | p = 0.0785 |
| Subjective | TST_{Full} Night 3 | 111.9 | 33.5 | 78.5 | p = 0.0225 |
| | TST_{Full} All 3 Nights | 240.0 | 65.1 | 174.9 | p = 0.0423 |
| | Sleep Quality Night 3 | 1.31 | 0.36 | 0.95 | p = 0.0198 |
| | Sleep Latency Night 3 | −20.6 | 6.0 | −26.5 | p = 0.0347 |
| | WASO Night 3 | −81.1 | −24.7 | −56.4 | p = 0.0840 |
| Global | PGI-S Day 3 | −0.71 | −0.07 | −0.63 | p = 0.0168 |
| measures | KSS Day 4 | −1.69 | −0.69 | −1.00 | p = 0.0765 |

As can be seen in Table 4, tasimelteon significantly improves both objective and subjective measures of sleep. Tasimelteon-treated patients sleep 76 minutes longer during the first ⅔ of Night 3 and a total of 131 minutes longer during the first ⅔ of all three nights during their treated travel as compared to their baseline observational travel.

Subjective measures of TST, sleep quality, sleep latency, and WASO made using a Post-Sleep Questionnaire (PSQ) demonstrate a similar improvement among tasimelteon-treated participants. Measures of global function, including Patient Global Impression of Severity (PGI-S) and KSS also favor tasimelteon.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

The corresponding structures, materials, acts, and equivalents of all means or step plus function elements in the claims below are intended to include any structure, material, or act for performing the function in combination with other claimed elements as specifically claimed. The description of the present disclosure is presented for purposes of illustration and description, but is not intended to be exhaustive or limited to the disclosure in the form disclosed. Many modifications and variations will be apparent to those of ordinary skill in the art without departing from the scope and spirit of the disclosure. Any embodiments chosen and described herein appear in order to best explain the principles of the disclosure and the practical application, and to enable others of ordinary skill in the art to understand the disclosure for various embodiments with various modifications as are suited to the particular use contemplated.

1. A method of treating an individual experiencing a sleep-wake-cycle-disrupting advance in the individual's established bedtime comprising administering to the individual an amount of tasimelteon effective to reduce one or more untoward consequences of the resulting disruption of said individual's established sleep-wake cycle.

2. The method of claim 1, wherein the advance in the individual's established bedtime is eight hours and caused by eastward jet aircraft travel.

3. The method of claim 2, wherein the amount of tasimelteon administered is 20 mg per day, administered for at least three consecutive days prior to bedtime, in an immediate release form, with the first dose administered prior to the first bedtime following the sleep-wake-cycle disrupting advance.

4. The method of claim 3, wherein the reduction of untoward consequences is an improvement in at least one sleep parameter selected from a group consisting of: total sleep time, latency to persistent sleep, and wake after sleep onset.

5. The method of claim 4, wherein the improved sleep parameter is total sleep time.

6. The method of claim 5, wherein total sleep time is improved in the first two-thirds of the night on the third night following tasimelteon administration.

7. The method of claim 3, wherein the reduction of untoward consequences is an improvement in next day alertness.

8. The method of claim 7, wherein the improvement in next day alertness includes an improvement on the Karolinska Sleepiness Scale, an improvement on the Visual Analog Scale, or both.

9. The method of claim 2, wherein the advance in the individual's established bedtime is six to eight hours and the amount of tasimelteon administered is 20 mg per day, administered in an immediate release form for three consecutive days prior to bedtime, with the first dose administered prior to the first bedtime following the sleep-wake-cycle disruption.

10. (canceled)

11. The method of claim 1, wherein the individual experiences the sleep-wake-cycle disrupting advance as a result of eastbound jet aircraft travel.

12. The method of claim 11, wherein the individual experiences a significantly improved total sleep time during the first two-thirds of the night, during the night following the administration of the third dose of tasimelteon, in both subjective and objective measures of sleep.

13. (canceled)

14. (canceled)

15. The method of claim 1, wherein the sleep-wake-cycle-disrupting advance occurs upon arrival, following an eastbound jet aircraft flight, in which clock time is advanced at the destination of up to eight hours relative to the clock time at the origin of the flight.

16-18. (canceled)

19. The method of claim 1, wherein the individual experiences an increase in total REM sleep, a reduction in the time to accumulation of 30 minutes of REM sleep, or both.

20. A method of treating jet lag in an individual subjected to eastward travel from a place of origin through six to eight time zones to a place of destination during overnight travel by air, said method comprising orally administering to the individual 20 mg tasimelteon in an immediate release form following arrival at the place of destination at or prior to the individual's bedtime in the place of destination.

21. The method of claim 20, wherein prior to the individual's bedtime in the place of destination means between 30 minutes and two hours prior to said individual's bedtime corresponding to the clock time of the individual's regular bedtime at his place of origin.

22. The method of claim 20, wherein prior to the individual's bedtime in the place of destination means within one and one-half hours of such bedtime.

23. (canceled)

24. (canceled)

25. The method of claim 20, wherein the individual is an individual who is pre-selected based on having suffered jet lag disorder as a result of previous similar travel experience.

26. The method of claim 20, wherein the individual is administered tasimelteon once daily for at least three days.

27-29. (canceled)

30. A pre-packaged dispensing unit comprising:
(a) a number of individual tasimelteon unit doses each containing 20 mg of immediate release tasimelteon sufficient to provide a course of treatment for an individual to be treated for a jet lag disorder; and
(b) a pharmaceutically acceptable container for said unit doses.

31. A dispensing unit of claim 30 in which the number of unit doses therein is 3, 4, or 5 and the container therefor is a blister pack containing one 20 mg tasimelteon tablet per blister.
